(19) United States
(12) Patent Application Publication
(10) Pub. No.: US 2012/0227749 A1
(43) Pub. Date: Sep. 13, 2012

(54) MOUTHPIECE

(75) Inventors: Yasuhiro Kondo, Okayama-shi (JP); Kazushi Kinugasa, Okayama-shi (JP); Chiho Honda, Okayama-shi (JP)

(73) Assignee: NATIONAL UNIVERSITY CORPORATION OKAYAMA UNIVERSITY, Okayama-shi, Okayama (JP)

(21) Appl. No.: 13/502,833

(22) PCT Filed: Sep. 14, 2010
(86) PCT No.: PCT/JP2010/005611

§ 371 Date: Apr. 19, 2012

(30) Foreign Application Priority Data
Oct. 20, 2009 (JP) 2009-241647

(51) Int. Cl. A61F 5/56

(52) U.S. Cl. 128/848

(57) ABSTRACT

Provided is a mouthpiece that does not come off easily even when a user suddenly moves, and that can alleviate snoring, apnea and symptoms of obstructive respiratory disorders. The mouthpiece has an upper piece (2) that fits to a row of teeth of an upper jaw (J1), a lower piece (3) that fits to a row of teeth of a lower jaw (J2), and a connecting member (4) that connects the upper and lower piece (2 and 3) so that the pieces are in a preset target position and so that the upper piece (2) and the lower piece (3) are relatively displaced while generating elastic force for returning to the target position by elastically deforming the connecting member according to an external force applied to at least one of the upper piece (2) and the lower piece (3).

### TECHNICAL FIELD

The present invention relates to a mouthpiece which is fitted in the oral cavity of a human body for inhibiting snoring and apnea during the treatment of obstructive respiratory disorders and during sleep.

### BACKGROUND ART

The cause of snoring and apnea during sleep is generally considered to be a result of the narrowing or obstruction of the air passage. Specifically, when the jaw muscles loosen during sleep in a supine position, the lower jaw of that person moves down (moves back relative to the human body), and it is considered that the air passage is consequently narrowed, and vibration of the soft palate or the like (snoring) occurs during breathing. In addition, when the backward movement of the lower jaw advances to an extent of obstructing the air passage, it is considered that symptoms of sleep apnea will occur.

Moreover, if the brain is damaged due to head injuries as represented in traffic accidents, it is known that similar circumstances; that is, so-called obstructive respiratory disorders, occur to patients of persistent disturbance of consciousness (so-called vegetative state).

In order to inhibit such snoring, sleep apnea and obstructive respiratory disorders, for example, known is an appliance that is fitted into an oral cavity of the human body as described in Unexamined Japanese Patent Publication No. H11-508472. The oral appliance of Unexamined Japanese Patent Publication No. H11-508472 comprises an upper shell including a groove for receiving an upper arched part of the human body, a lower shell including a groove for covering a lower arched part of the human body, a ring provided downward to the upper shell, and an arm protruding upward from the lower shell, wherein the shells are connected as a result of the arm being introduced into the ring. The lower shell is biased toward the upper shell so as to thrust the lower jaw forward based on the spring effect of the arm.

Nevertheless, with the oral appliance of Unexamined Japanese Patent Publication No. H11-508472, since the upper shell and the lower shell configured separately are connected in a state where the lower shell is biased toward the upper shell so that it is thrust forward, there is a problem in that the position of the lower shell relative to the upper shell during the fitting cannot be fixed to a target position.

### SUMMARY OF THE INVENTION

An object of the present invention is to provide a mouthpiece capable of fixing the user's upper jaw and lower jaw to a target position, which does not come off easily even when the user suddenly moves, and which can alleviate snoring, apnea and symptoms of obstructive respiratory disorders.

In order to achieve the foregoing object, the present invention provides a mouthpiece comprising an upper piece which can be fitted to at least a part of a row of teeth of an upper jaw, a lower piece which can be fitted to at least a part of a row of teeth of a lower jaw, and at least one connecting member which is provided between the upper piece and the lower piece, connects the upper piece and the lower piece to each other so that the pieces are in a target position that is preset as a position of a front-and-back direction of the upper piece and the lower piece, and which generates elastic force for returning to the target position by elastically deforming the connecting member according to an external force applied to at least one of the upper piece and the lower piece, wherein the connecting member is configured from a spring including a coil part wound around an axis in an up-and-down direction, a first end guided upward from the coil part, and a second end guided downward from the coil part, and wherein, in a state where the coil part is sandwiched between the upper piece and the lower piece, the first end is fixed to the upper piece and the second end is fixed to the lower piece, respectively.

### BRIEF DESCRIPTION OF THE DRAWINGS

FIG. 1 is a perspective view showing the overall configuration of the mouthpiece according to an embodiment of the present invention.

FIG. 2 is a side view of the mouthpiece of FIG. 1.

FIG. 3 is a bottom view of the mouthpiece of FIG. 1 in a state where the lower piece is omitted.

FIG. 4 is an arrow view of IV of FIG. 3.

FIG. 5 is diagram schematically showing the row of teeth of the upper jaw and the lower jaw of a human being.

FIG. 6 is a schematic diagram showing the positional relationship of the upper jaw and the lower jaw which may cause snoring, apnea and symptoms of obstructive respiratory disorders when a person is sleeping in a supine position.

FIG. 7 is a schematic diagram showing a state where the lower jaw is thrust forward from the state of FIG. 6.

### BEST MODE FOR CARRYING OUT THE INVENTION

Preferred embodiments of the present invention are now explained with reference to the appended drawings.

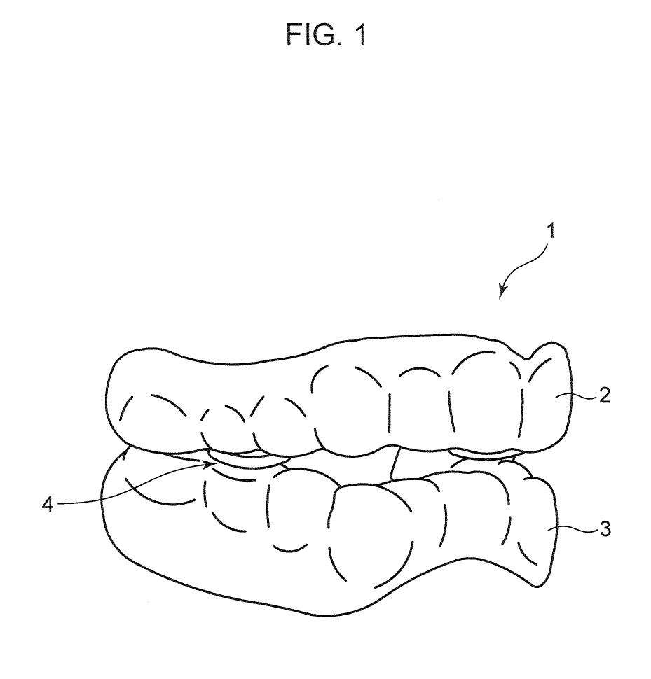

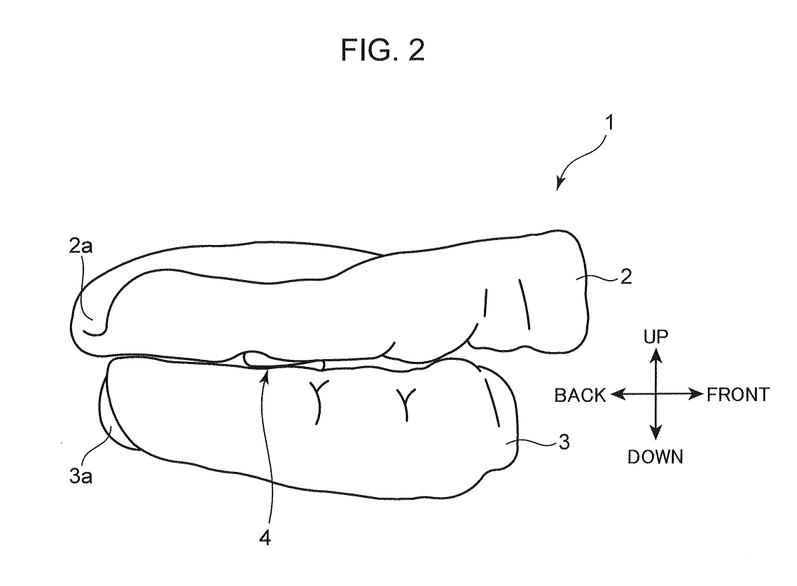

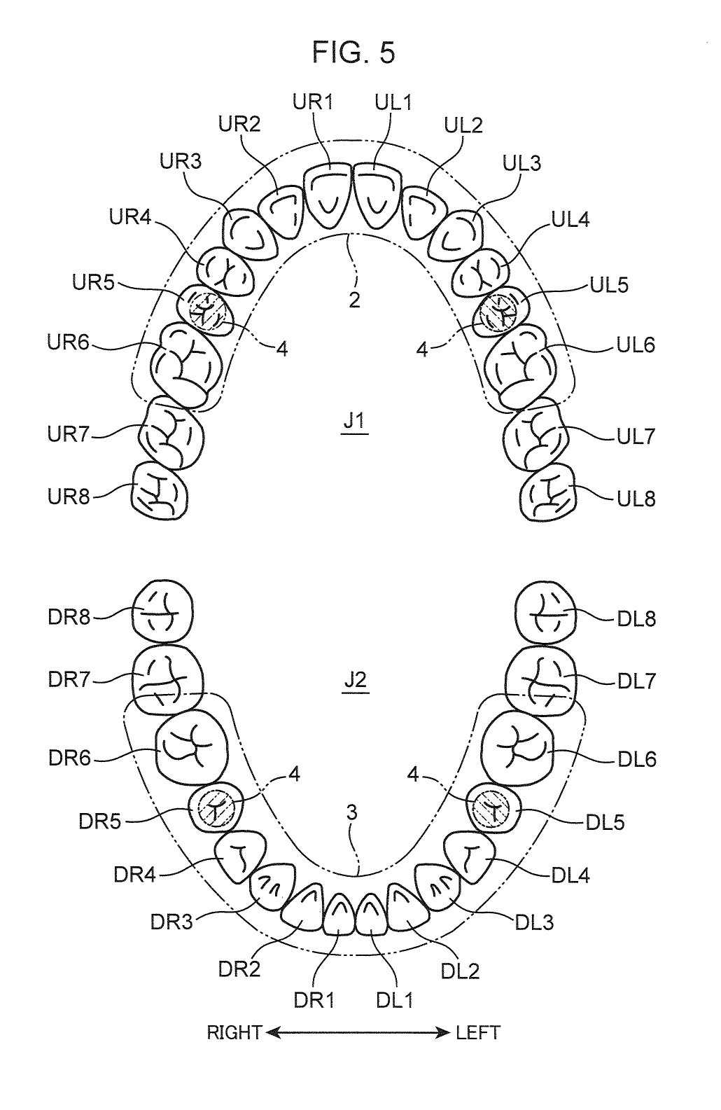

FIG. 1 is a perspective view showing the overall configuration of the mouthpiece according to an embodiment of the present invention. FIG. 2 is a side view of the mouthpiece of FIG. 1. FIG. 5 is diagram schematically showing the row of teeth of the upper jaw and the lower jaw of a human being. Note that the ensuing explanation is provided by using the up-and-down, right-and-left and front-and-back directions in a state where the mouthpiece of FIG. 1 and FIG. 2 is fitted in the oral cavity of a human being.

Referring to FIG. 1, FIG. 2 and FIG. 5, the mouthpiece 1 comprises an upper piece 2 which can be fitted to a row of teeth of a person's upper jaw J1, a lower piece 3 which can be fitted to a row of teeth of a person's lower jaw J2, and a connecting member 4 for connecting the upper piece 2 and the lower piece 3.

The upper piece 2 is a member made of synthetic resin (for instance, PET [Polyethyleneterephthalate]) formed in a U-shape (refer to FIG. 5) in a planar view so as to cover the row of teeth of the upper jaw J1. Specifically, the upper piece 2 includes, as shown in FIG. 5, a groove part 2a (refer to FIG. 2) for accepting teeth from a right molar UR6 to a left molar UL6 of the row of teeth of the upper jaw J1. In order to prepare this upper piece 2, a female die of the patient's row of teeth is taken, and this female die is used to obtain a plaster die (male die). In addition, as a result of pressing molten resin to the plaster die, it is possible to obtain the upper piece 2 capable of covering the plaster die. Here, in order to increase the retaining force of the upper piece 2 relative to the row of teeth upon fitting it to the row of teeth, preferably, a groove part 2a having a depth capable of accept the respective teeth up to the root of their tooth crown part is formed. Moreover, it is also possible to form a portion that is more prominent than the other portions on the lateral surface (surface facing the right-and-left direction and front direction) of the upper piece 2, and use such prominent part as the gripping portion (portion for hooking a finger or the like) upon fitting and removing the upper piece 2. If the prominent part is insufficient for the fitting and removal of the upper piece 2, quick polymerize-type acrylic resin (for example, MMA-PMMA [Methylmethacrylate]) may be added to correct the shape.

The lower piece 3 is a member made of synthetic resin (for instance, PET [Polyethyleneterephthalate]) formed in a U-shape (refer to FIG. 5) in a planar view so as to cover the row of teeth of the lower jaw J2. Specifically, the lower piece 3 includes, as shown in FIG. 5, a groove part 3a (refer to FIG. 2) for accepting teeth from a right molar DR6 to a left molar DL6 of the row of teeth of the lower jaw J2. In order to prepare this lower piece 3, as with the foregoing upper piece 2, a female die of the patient's row of teeth is taken, and this female die is used to obtain a plaster die (male die). In addition, as a result of pressing molten resin to the plaster die, it is possible to obtain the lower piece 3 capable of covering the plaster die. As with the foregoing upper piece 2, preferably a groove part 3a having a depth capable of accept the respective teeth up to the root of their tooth crown part is also formed in the lower piece 3. Moreover, as with the foregoing upper piece 2, an prominent part can also be formed on the lateral surface of the lower piece 3.

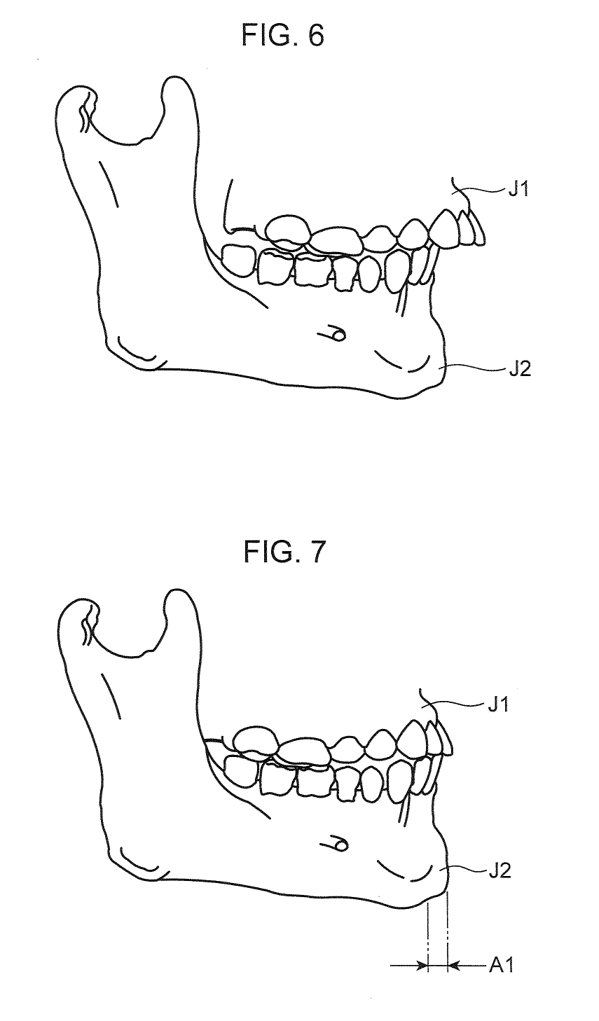

The upper piece 2 and the lower piece 3 configured as described above are mutually connected by the connecting member 4 described later so that the pieces are in a preset target position. The target position is foremost explained with reference to FIG. 6 and FIG. 7.

FIG. 6 is a schematic diagram showing the positional relationship of the upper jaw and the lower jaw which may cause snoring, apnea and symptoms of obstructive respiratory disorders when a person is sleeping in a supine position. FIG. 7 is a schematic diagram showing a state where the lower jaw is thrust forward from the state of FIG. 6.

In the state of FIG. 6, since jaw muscles loosen, the lower jaw J2 moves down (moves back relative to the human body) from the upper jaw J1, and the air passage not shown is narrowed or obstructed. In this state, there is a possibility that snoring, apnea or symptoms of obstructive respiratory disorders will occur. Meanwhile, in the state of FIG. 7, the narrowing or obstruction of the air passage is alleviated since the lower jaw J2 is positioned forward by a length A1 in comparison to the state of FIG. 6. Here, it has been confirmed that the air passage can be secured by setting the length A1 to 1 cm or longer in healthy human body. In this embodiment, the position of the upper piece 2 and the lower piece 3 in the front-and-back direction, in which the positional relationship of the upper jaw J1 and the lower jaw J2 becomes the relationship shown in FIG. 7, is set as the target position. The connecting member 4 explained below connects the upper piece 2 and the lower piece 3 so that they are in a position corresponding to the target position.

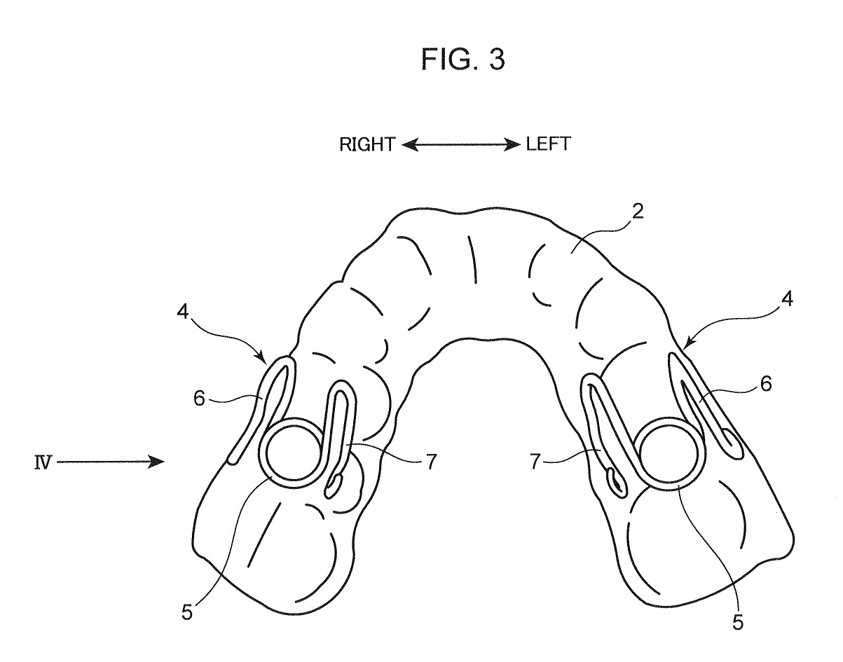

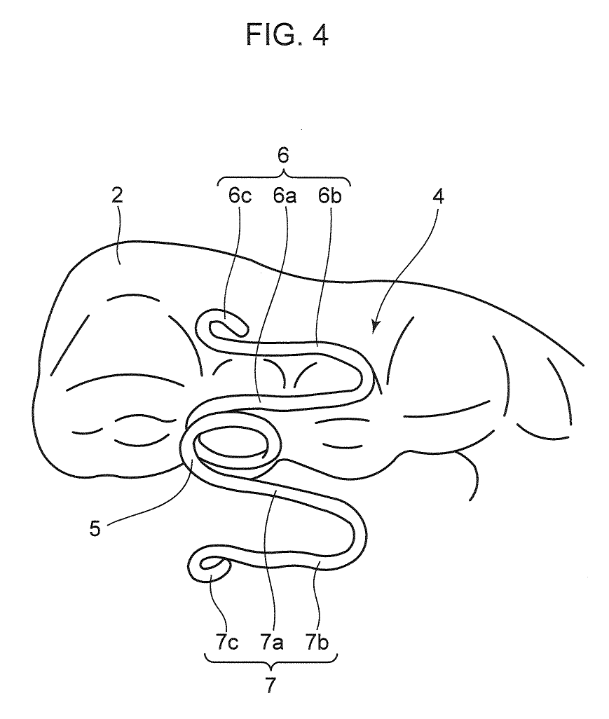

FIG. 3 is a bottom view of the mouthpiece 1 of FIG. 1 in a state where the lower piece 3 is omitted. FIG. 4 is an arrow view of IV of FIG. 3.

Referring to FIG. 3 to FIG. 5, a total of two connecting members 4 is provided, one of the connecting members 4 is provided between a premolar UR5 of the upper jaw J1 and a premolar DR5 of the lower jaw J2, and the other of the connecting members 4 is provided between a premolar UL5 of the upper jaw J1 and a premolar DL5 of the lower jaw J2. The connecting members 4 of this embodiment are configured from a spring formed of cobalt-chromium alloy wiring. Specifically, the connecting member 4 comprises a coil part 5 disposed between the upper piece 2 and the lower piece 3, a first end 6 guided from the coil part 5 and extending upward, and a second end 7 guided from the coil part 5 and extending downward.

The coil part 5 is configured by the cobalt-chromium alloy wiring being wound around an axis in a up-and-down direction (a full circle and a half in this embodiment). Specifically, as shown in FIG. 3, with the right-side connecting member 4, the cobalt-chromium alloy wiring is wound upward in the clockwise direction when viewed from below, and with the left-side connecting member 4, the cobalt-chromium alloy wiring is wound upward in the counterclockwise direction when viewed from below.

The first end 6 includes, as shown in FIG. 4, a forward tilting part 6a extending from the coil part 5 obliquely upward at a forward tilt, a backward tilting part 6b extending from the forward tilting part 6a in a U-turn and obliquely upward at a backward tilt, and a loop part 6c which makes a further U-turn from the backward tilting part 6b and forms a loop at the front thereof. As shown in FIG. 3, the first end 6 of the right-side connecting member 4 is guided to the right side of the coil part 5 and fixed to the right side face (lateral surface) of the upper piece 2. Meanwhile, the first end 6 of the left-side connecting member 4 is guided to the left side of the coil part 5 and fixed to the left side face (lateral surface) of the upper piece 2.

The fixation of the first end 6 and the lateral surface of the upper piece 2 can be performed as follows. Foremost, in a state where the first end 6 is closely attached to the lateral surface of the upper piece 2, quick polymerization-type acrylic resin (for instance, MMA-PMMA [Methylmethacrylate]) is applied from the outside of the first end 6 onto the lateral surface of the upper piece 2. When the quick polymerization-type acrylic resin is polymerized, the first end 6 becomes fixed in a state of being embedded in the resin, and, consequently, the first end 6 and the upper piece 2 can be fixed. In this embodiment, since the first end 6 of a bent shape including the forward tilting part 6a, the backward tilting part 6b, and the loop part 6c as described above is adopted, it is possible to lengthen the fixed length (distance) of the first end 6 relative to the upper piece 2, and, consequently, the fixation strength can be increased in comparison to the case of adopting a linear first end 6.

The second end 7 includes, as shown in FIG. 4, a forward tilting part 7a extending from the coil part 5 obliquely downward at a forward tilt, a backward tilting part 7b extending from the forward tilting part 7a in a U-turn and obliquely downward at a backward tilt, and a loop part 7c which makes a further U-turn from the backward tilting part 7b and forms a loop at the front thereof. Note that, in FIG. 3, the lower piece 3 is omitted, but the second end 7 of the right-side connecting member 4 is guided to the left side of the coil part 5 and fixed to the left side face (internal surface) of the lower piece 3. Meanwhile, the second end 7 of the left-side connecting member 4 is guided to the right side of the coil part 5 and fixed to the right side face (internal surface) of the lower piece 3. The fixation of the second end 7 and the internal surface of the lower piece 3 can be performed with the same method of fixing the first end 6 and the upper piece 2 described above.

In addition, with the mouthpiece 1 of this embodiment, as shown in FIG. 2, the upper piece 2 and the lower piece 3 are connected via only the connecting member 4. In other words, the upper piece 2 and the lower piece 3 are not connected at portions other than the connecting member 4.

The operation of the mouthpiece 1 of this embodiment is now explained.

In this embodiment, axis line direction of the winding of the connecting member 4 is set to the up-and-down direction, and both ends 6, 7 are respectively fixed to the upper piece 2 and the lower piece 3. Thus, the upper piece 2 and the lower piece 3 can be separated in the up-and-down direction according to the extension of the coil part 5, and the upper piece 2 and the lower piece 3 can return to their normal position based on the contraction of the coil part 5.

Moreover, the axis line direction of the winding of the coil part 5 of the connecting member 4 is set to the up-and-down direction, and the first end 6 and the second end 7 guided in the right-and-left direction from the coil part 5 are fixed to the upper piece 2 and the lower piece 3. Thus, the first end 6 and the second end 7 can move in the front-and-back direction and the right-and-left direction together with the deformation of the coil part 5; that is, in a direction of the coil part 5 being narrowed or in a direction of the coil part 5 being opened. Consequently, the relative displacement of the upper piece 2 and the lower piece 3 can also be tolerated in the front-and-back and right-and-left directions and the turning direction.

In addition, the connecting members 4 is provided in a pair in a right-and-left direction, and the first end 6 and the second end 7 guided from the coil part 5 of the connecting members 4 in opposite directions of the right-and-left direction are fixed to the upper piece 2 and the lower piece 3. Thus, the turning of the front part of the upper piece 2 and the lower piece 3 around the respective connecting members 4 (closing and opening of the user's front teeth) is allowed.

In addition, by fitting the upper piece 2 to the user's upper jaw J1 and fitting the lower piece 3 to the user's lower jaw J2, the positional relationship of the user's upper jaw J1 and lower jaw J2 are corrected to a front-and-back position of the upper piece 2 and the lower piece 3 defined by the connecting member 4; that is, the target position shown in FIG. 7.

If the user suddenly moves such as coughing, choking, sneezing or swallowing in the foregoing fitted state, the upper jaw J1 and the lower jaw J2 will be relatively displaced in the up-and-down, front-and-back and right-and-left directions or a combined direction thereof. Here, the mouthpiece 1 of this embodiment can tolerate the relative displacement of the upper piece 2 and the lower piece 3 in the up-and-down, right-and-left and front-and-back directions while generating elastic force for returning to the target position by elastically deforming the coil part 5 as described above. Accordingly, even if the user suddenly moves, the mouthpiece 1 does not easily come off from the upper jaw J1 and the lower jaw J2.

As explained above, according to the mouthpiece 1, since the upper piece 2 and the lower piece 3 are connected in a state of being positioned at the target position by the connecting member 4, the positional relationship of the user's upper jaw J1 and lower jaw J2 can be corrected to a positional relationship shown in FIG. 7 (positional relationship capable of securing the air passage). Thus, according to the mouthpiece 1, it is possible to secure the air passage of the person wearing the mouthpiece 1, and it is thereby possible to alleviate snoring, apnea and symptoms of obstructive respiratory disorders.

In addition, with the mouthpiece 1, since the upper piece 2 and the lower piece 3 are connected by an elastically deformable connecting member 4, even if the person wearing the mouthpiece suddenly moves (for example, coughing, choking, sneezing or swallowing), the mouthpiece 1 will not easily come off the upper jaw J1 or the lower jaw J2. Specifically, since the connecting member 4 (coil part 5) will elastically deform according to the external force applied to the upper piece 2 or the lower piece 3, the relative displacement of the upper piece 2 and the lower piece 3 is allowed. Thus, even if there is a sudden movement that causes the relative displacement of the upper piece 2 and the lower piece 3, the mouthpiece 1 will not come off easily from the upper jaw J1 or the lower jaw J2. In addition, after the foregoing sudden movement, the upper piece 2 and the lower piece 3 return to the target position based on the elastic force of the connecting member 4 (coil part 5) that was generated by the relative displacement of the upper piece 2 and the lower piece 3, and it is thereby possible to alleviate snoring, apnea and symptoms of obstructive respiratory disorders.

According to the mouthpiece 1 including the connecting member 4 configured from a spring including the coil part 5 and the first end 6 and the second end 7, since the first end 6 is fixed to the upper piece 2 and the second end 7 is fixed to the lower piece 3, respectively, the upper piece 2 and the lower piece 3 are positioned at the target position. In addition, according to the mouthpiece 1, since the coil part 5 wound around an axis in the up-and-down direction is sandwiched between the upper piece 2 and the lower piece 3, the relative up-and-down motion of the upper piece 2 and the lower piece 3 is allowed based on the expansion and contraction of the coil part 5. Moreover, when both ends 6, 7 operate in the direction of squeezing the coil part 5 or in the direction of opening the coil part 5, the relative displacement of the upper piece 2 and the lower piece 3 is allowed on a plane along the front-and-back direction and the right-and-left direction. In addition, as a result of the coil part 5 operating in its turning direction, the relative displacement of the upper piece 2 and the lower piece 3 in the turning direction around the axis along the up-and-down direction is allowed. Thus, according to the mouthpiece 1, provided is a mouthpiece 1 which will not come off easily even when the user suddenly moves as a result of allowing the relative displacement of the upper piece 2 and the lower piece 3 in the up-and-down, right-and-left, front-and-back and turning directions. Moreover, if both ends 6, 7 of the connecting member 4 are respectively fixed to the upper piece 2 and the lower piece 3 as with the mouthpiece 1, the ends of the connecting member 4 will not be disposed between the upper piece 2 and the lower piece 3 and, therefore, it is possible to reduce the possibility of the ends 6, 7 of the connecting member 4 coming into contact with the oral tissue when the user wears the mouthpiece 1.

When the connecting member 4 is fixed to the upper piece 2 and the lower piece 3 at a position on both sides of the coil part 5 in the right-and-left direction via the first end 6 and the second end 7 guided from the coil part 5 in opposite direction of the right-and-left direction as in this embodiment, since the first end 6 and the second end 7 are connected to the upper piece 2 and the lower piece 3 at a position on both sides of the coil part 5, the operation of the first end 6 and the second end 7 of trying to flex the coil part 5 in the front-and-back direction can be performed easily. Consequently, the turning of the front part of the upper piece 2 and the front part of the lower piece 3 around the connecting member 4 (closing and opening of the user's front teeth) is allowed. Accordingly, since it is possible to inhibit a state where the user's mouth remains open, this contributes to inhibiting the inside of the user's oral cavity from becoming dry.

When the first end 6 is fixed to the lateral surface of the upper piece 2 and the second end 7 is fixed to the internal surface of the lower piece 3 as in this embodiment, by disposing the first end 6 and the second end 7 on the side of the upper piece 2 and the lower piece 3, the gap in the up-and-down direction between the upper piece 2 and the lower piece 3 can be narrowed. In other words, with the mouthpiece 1, in comparison to a case of inserting the entire connecting member 4 between the upper piece 2 and the lower piece 3, the gap between the upper piece 2 and the lower piece 3 can be narrowed by disposing the first end 6 and the second end 7 on the side of the upper piece 2 and the lower piece 3. It is thereby possible to narrow the range in which the user's mouth is opened upon wearing the mouthpiece and inhibit the drying of the user's mouth. In addition, as a result of using the side face of the upper piece 2 and the lower piece 3 as the space for fixing the first end 6 and the second end 7, the strength of fixing both ends 6, 7 can be sufficiently secured.

Note that, in this embodiment, although the first end 6 is fixed to the lateral surface of the upper piece 2 and the second end 7 is fixed to the internal surface of the lower piece 3, the same effects as those described above can be obtained by fixing the first end 6 to the internal surface of the upper piece 2 and fixing the second end 7 to the lateral surface of the lower piece 3.

When the upper piece 2 and the lower piece 3 are provided to cover teeth from the right molars UR6, DR6 to the left molars UL6, DL6 and connecting members 4 is provided in a pair in the right-and-left direction as in this embodiment, the relative displacement of the upper piece 2 and the lower piece 3 can be performed in a well-balanced manner in the right-and-left direction by causing the two connecting members 4 to elastically deform.

Note that, in this embodiment, the upper piece 2 and the lower piece 3 that are provided to cover teeth from the right molars UR6, DR6 to the left molars UL6, DL6 are adopted, but the foregoing effect of alleviating snoring, sleep apnea and obstructive respiratory disorders can be obtained if the upper piece 2 and the lower piece 3 can cover teeth up to at least the first molar.

When the connecting member 4 is provided respectively between the premolar UR5 and the premolar DR5 and between the premolar UL5 and the premolar DL5 as in this embodiment, as a result of disposing the respective connecting members 4 near the jaw joint, the turning of the front part of the upper piece 2 and the front part of the lower piece 3 around the respective connecting members 4 (closing and opening of the user's front teeth) is allowed. Accordingly, since it is possible to inhibit a state where the user's mouth remains open, this contributes to inhibiting the inside of the user's oral cavity from becoming dry.

Note that the foregoing effect can be obtained so as long as the connecting members 4 are respectively provided within a range of at least from the left and right first premolars to the first molars.

Moreover, when the respective connecting members 4 are disposed near the jaw joint as with the mouthpiece 1, the external force that is applied to the connecting member 4 during use can be reduced. In other words, since the distance of the relative rotational motion of the upper jaw J1 and the lower jaw J2 around the jaw joint will be shorter as the connecting members 4 are disposed closer to the jaw joint, the amount of elastic deformation of the connecting members 4 can be reduced by providing the respective connecting members 4 to the back teeth as with the mouthpiece 1, and the load on the connecting member 4 can be reduced. From the user's perspective, since it is possible to reduce the force that must be applied to the upper piece 2 and the lower piece 3 upon attempting to maintain the user's desired closing/opening position of the upper jaw J1 and the lower jaw J2, this means that the load on the user can also be reduced.

While the mouthpiece 1 of this embodiment comprises two connecting members 4, at least one connecting member 4 needs to be provided in order to fix the user's upper jaw and lower jaw to the target position, realize a mouthpiece that does not come off easy even when the user suddenly moves, and alleviate snoring, sleep apnea and symptoms of obstructive respiratory disorders.

Note that the specific embodiments described above mainly include the invention configured as described below.

The mouthpiece according to this embodiment comprises an upper piece which can be fitted to at least a part of a row of teeth of an upper jaw, a lower piece which can be fitted to at least a part of a row of teeth of a lower jaw, and at least one connecting member which is provided between the upper piece and the lower piece, connects the upper piece and the lower piece to each other so that the pieces are in a target position that is preset as a position of a front-and-back direction of the upper piece and the lower piece, and which generates elastic force for returning to the target position by elastically deforming the connecting member according to an external force applied to at least one of the upper piece and the lower piece, wherein the connecting member is configured from a spring including a coil part wound around an axis in an up-and-down direction, a first end guided upward from the coil part, and a second end guided downward from the coil part, and wherein, in a state where the coil part is sandwiched between the upper piece and the lower piece, the first end is fixed to the upper piece and the second end is fixed to the lower piece, respectively.

According to the mouthpiece of this embodiment, since the upper piece and the lower piece are connected in a state of being positioned at the target position by the connecting member, the positional relationship of the user's upper jaw and lower jaw can be corrected to a position that corresponds to the target position. Accordingly, by setting the target position to a position for securing the air passage during sleep; specifically, a position of causing the lower jaw of the person sleeping in a supine position to be thrust forward relative to the upper jaw, it is possible to secure the air passage of the person wearing the mouthpiece, and it is thereby possible to alleviate snoring, apnea and symptoms of obstructive respiratory disorders. Note that, with the conventional oral appliance (Unexamined Japanese Patent Publication No. H11-508472), while the position of the upper shell and the lower shell will become steady at a well-balanced position of the force of the user's jaw and the spring effect of the arm, this position is not a position that is preset as the positional relationship of the upper jaw and the lower jaw, and is a position that differs on a case-by-case basis.

In addition, with the mouthpiece according to this embodiment, since the upper piece and the lower piece are connected by an elastically deformable connecting member, even if the person wearing the mouthpiece suddenly moves (for example, coughing, choking, sneezing or swallowing), the mouthpiece will not easily come off the upper jaw or the lower jaw. Specifically, since the connecting member will elastically deform according to the external force applied to the upper piece or the lower piece, the relative displacement of the upper piece and the lower piece is allowed. Thus, even if there is a sudden movement that causes the relative displacement of the upper piece and the lower piece, the mouthpiece will not come off easily from the upper jaw or the lower jaw. In addition, after the foregoing sudden movement, the upper piece and the lower piece return to the target position based on the elastic force of the connecting member that was generated by the relative displacement of the upper piece and the lower piece, and it is thereby possible to alleviate snoring, sleep apnea and symptoms of obstructive respiratory disorders.

Moreover, according to the mouthpiece of this embodiment, since the first end is fixed to the upper piece and the second end is fixed to the lower piece, respectively, the upper piece and the lower piece are positioned at the target position. In addition, according to the mouthpiece of this embodiment, since the coil part wound around an axis in the up-and-down direction is sandwiched between the upper piece and the lower piece, the relative up-and-down motion of the upper piece and the lower piece is allowed based on the expansion and contraction of the coil part. Moreover, when both ends operate in the direction of squeezing the coil part or in the direction of opening the coil part, the relative displacement of the upper piece and the lower piece is allowed on the plane along the front-and-back direction and a right-and-left direction. In addition, as a result of the coil part operating in its turning direction, the relative displacement of the upper piece and the lower piece in the turning direction around the axis along the up-and-down direction is allowed. Thus, according to the mouthpiece of this embodiment, provided is a mouthpiece which will not come off easily even when the user suddenly moves as a result of allowing the relative displacement of the upper piece and the lower piece in the up-and-down direction, the right-and-left direction, the front-and-back direction and the turning directions. Moreover, if both ends of the connecting member are respectively fixed to the upper piece and the lower piece as with the mouthpiece of this embodiment, the ends of the connecting member will not be disposed between the upper piece and the lower piece and, therefore, it is possible to reduce the possibility of the ends of the connecting member coming into contact with the oral tissue when the user wears the mouthpiece.

In the foregoing mouthpiece, preferably, the first end and the second end are guided from the coil part in opposite directions of a right-and-left direction respectively, and the connecting member is fixed to the upper piece and the lower piece via the first end and the second end at a position on both sides of the coil part in the right-and-left direction.

According to the foregoing mouthpiece, since the first end and the second end are connected to the upper piece and the lower piece at a position on both sides of the coil part in the right-and-left direction, the operation of the first end and the second end of trying to flex the coil part in the front-and-back direction can be performed easily. Consequently, the turning of the front part of the upper piece and the front part of the lower piece around the connecting member (closing and opening of the user's front teeth) is allowed. Accordingly, since it is possible to inhibit a state where the user's mouth remains open, this contributes to inhibiting the inside of the user's oral cavity from becoming dry.

In the foregoing mouthpiece, preferably, the first end is fixed to one of a right side face and a left side face of the upper piece, and the second end is fixed to the other one of the right side face and the left side face of the lower piece.

According to the foregoing mouthpiece, by disposing the first end and the second end on the side of the upper piece and the lower piece, the gap in the up-and-down direction between the upper piece and the lower piece can be narrowed. In other words, with the foregoing mouthpiece, in comparison to a case of inserting the entire connecting member between the upper piece and the lower piece, the gap between the upper piece and the lower piece can be narrowed by disposing the first end and the second end on the side of the upper piece and the lower piece. It is thereby possible to narrow the range in which the user's mouth is opened upon wearing the mouthpiece and inhibit the drying of the user's mouth. In addition, as a result of using the side face of the upper piece and the lower piece as the space for fixing the first end and the second end, the strength of fixing both ends can be sufficiently secured.

In the foregoing mouthpiece, preferably, the upper piece and the lower piece are respectively configured so that these pieces can be fitted at least to a range from right back teeth to left back teeth in the row of teeth, and the connecting member is provided in a pair in a right-and-left direction.

According to the foregoing mouthpiece, since the upper piece and the lower piece are provided to a range between the left and right back teeth and connecting members is provided in a pair in the right-and-left direction, the relative displacement of the upper piece and the lower piece can be performed in a well-balanced manner in the right-and-left direction by causing the two connecting members to elastically deform.

Note that the term “back teeth” means the teeth that are located farther distal (back) than at least the first or second molar.

In the foregoing mouthpiece, preferably, each of the connecting members is provided at a position corresponding to the left and right back teeth.

According to the foregoing mouthpiece, as a result of disposing the respective connecting members near the jaw joint, the turning of the front part of the upper piece and the front part of the lower piece around the connecting member (closing and opening of the user's front teeth) is allowed. Accordingly, since it is possible to inhibit a state where the user's mouth remains open, this contributes to inhibiting the inside of the user's oral cavity from becoming dry.

Moreover, when the respective connecting members are disposed near the jaw joint as with the foregoing mouthpiece, the external force that is applied to the connecting member during use can be reduced. In other words, since the distance of the relative rotational motion of the upper jaw and the lower jaw around the jaw joint will be shorter as the connecting members are disposed closer to the jaw joint, the amount of elastic deformation of the connecting members can be reduced by providing the respective connecting members to the back teeth as with the foregoing mouthpiece, and the load on the connecting member can be reduced. From the user's perspective, since it is possible to reduce the force that must be applied to the upper piece and the lower piece upon attempting to maintain the user's desired closing/opening position of the upper jaw and the lower jaw, this means that the load on the user can also be reduced.

Note that the term “back teeth” where the connecting members are to be disposed need to be within the range of the second premolar to the first molar in order to effectively realize the relative motion of the upper piece and the lower piece.

Moreover, the mouthpiece according to this embodiment comprises an upper piece which can be fitted to at least a part of a row of teeth of an upper jaw, a lower piece which can be fitted to at least a part of a row of teeth of a lower jaw, and at least one connecting member which is provided between the upper piece and the lower piece and connects the upper piece and the lower piece so that the pieces are in a target position that is preset as a position of a front-and-back direction of the upper piece and the lower piece, wherein the connecting member generates elastic force for returning to the target position by elastically deforming the connecting member according to an external force applied to at least one of the upper piece and the lower piece.

In the foregoing mouthpiece, preferably, the connecting member is configured from a spring including a coil part wound around an axis in an up-and-down direction, a first end guided upward from the coil part, and a second end guided downward from the coil part, and, in a state where the coil part is sandwiched between the upper piece and the lower piece, the first end is fixed to the upper piece and the second end is fixed to the lower piece, respectively.

### INDUSTRIAL APPLICABILITY

According to the mouthpiece of the present invention, since it is possible to follow the sudden movement of the user's lower jaw, the mouthpiece does not come off easily from the user, and can alleviate snoring, sleep apnea and symptoms of obstructive respiratory disorders.

1. A mouthpiece, comprising:
an upper piece which can be fitted to at least a part of a row of teeth of an upper jaw;
a lower piece which can be fitted to at least a part of a row of teeth of a lower jaw; and
at least one connecting member which is provided between the upper piece and the lower piece, connects the upper piece and the lower piece to each other so that the pieces are in a target position that is preset as a position of a front-and-back direction of the upper piece and the lower piece, and which generates elastic force for returning to the target position by elastically deforming the connecting member according to an external force applied to at least one of the upper piece and the lower piece, wherein
the connecting member is configured from a spring including a coil part wound around an axis in an up-and-down direction, a first end guided upward from the coil part, and a second end guided downward from the coil part, and
in a state where the coil part is sandwiched between the upper piece and the lower piece, the first end is fixed to the upper piece and the second end is fixed to the lower piece, respectively.

2. The mouthpiece according to claim 1, wherein
the first end and the second end are guided from the coil part in opposite directions of a right-and-left direction respectively, and
the connecting member is fixed to the upper piece and the lower piece via the first end and the second end at a position on both sides of the coil part in the right-and-left direction.

3. The mouthpiece according to claim 1, wherein
the first end is fixed to one of a right side face and a left side face of the upper piece, and
the second end is fixed to the other one of the right side face and the left side face of the lower piece.

4. The mouthpiece according to claim 1, wherein
the upper piece and the lower piece are respectively configured so that these pieces can be fitted at least to a range from right back teeth to left back teeth in the row of teeth, and
the connecting member is provided in a pair in a right-and-left direction.

5. The mouthpiece according to claim 4, wherein each of the connecting members is provided at a position corresponding to the left and right back teeth.
